(19) United States
(12) Patent Application Publication
(10) Pub. No.: US 2007/0197573 A1
(43) Pub. Date: Aug. 23, 2007

(54) Compositions and methods in the treatment of bone metabolic disorders

(76) Inventors: Wolfgang Sadee, Columbus, OH (US); Edward J. Bilsky, Biddeford, ME (US)

Correspondence Address: MUELLER AND SMITH, LPA;MUELLER-SMITH BUILDING, 7700 RIVERS EDGE DRIVE, COLUMBUS, OH, 43235, US

(21) Appl. No.: 11/542,711

(22) Filed: Oct. 4, 2006

Related U.S. Application Data
(60) Provisional application No. 60/723,502, filed on Oct. 4, 2005.

(51) Int. Cl. A61K 31/485

(52) U.S. Cl. 514/282

(57) ABSTRACT

Bone metabolic disorders are treated by administering to an individual a therapeutically effective amount of a peripheral opioid antagonist at one or more of the opioid receptors, including the various naloxone and naltrexone analogs or a pharmaceutically acceptable salt thereof. The invention is further embodied in the use of peripheral antagonists of the opioid receptors, such as the use of naltrexone and naloxone analogs, which can be opioid antagonist with peripheral selectivity at the μ opioid receptor, for the treatment of bone loss, osteoporosis, osteopenia and other bone disorders in individuals using opioid drugs, including patients using opioids for analgesia and in opioid drug-dependent individuals

### CROSS-REFERENCE TO RELATED APPLICATIONS

This application claims the benefit of pending U.S. Provisional Patent Application Ser. No. 60/723,502, filed Oct. 4, 2005, the disclosure of which is hereby incorporated by reference.

### STATEMENT REGARDING FEDERALLY SPONSORED RESEARCH

Not applicable.

### BACKGROUND OF THE INVENTION

Endogenous opiate receptors were discovered in the 1970s, and have been intensely studied in relation to seeking the mechanisms by which use of opiate drugs leads to addiction. Nonetheless, the molecular mechanisms leading to addiction have remained elusive. See, for example, J. Neurosci., 12(7): 2349-2450 (1992). In this past research, much of the focus has been on the interaction between opiate receptors and pain management and the euphoria that may accompany opiate use.

Recently, molecular studies of the mechanism of action of opiates have revealed structural components of opioid activity. A number of different opioid receptor types have been identified. Known receptor types include, for example, the mu—(μ, MOR), delta—(∂, DOR), and kappa—(k, KOR) receptors. Narcotic analgesics act at the opioid k-receptor to produce analgesia and dysphoria. The mu-receptor mediates analgesia, respiratory depression, and inhibition of gastrointestinal transit. As such, narcotic analgesics act at the delta-receptor to produce analgesia. However, continued use of narcotic analgesics typically leads to habit or addiction, and use of one leads to cross-tolerance/dependence for the others. Despite their therapeutic uses, undesirable side effects such as physical dependence and drug craving can develop.

It is recognized in the art that the native opioid receptors interact with endogenous opioid peptides, that are the natural ligands for the opioid receptors. Certain such ligands are the enkephalins, which are generally known to be pentapeptides. Another set of opioid receptor binding ligands are the endorphins (endogenous morphine analogs, e.g., β-endorphin) and the dynorphins. All of the endogenous opioid peptides along with the three major classes of opioid receptors are believed to be involved in the modulation of pain, including by modulating activity at the synaptic level. The central nervous system, including particularly the brain, is relatively rich in opioid receptors. Certain opioid receptor ligands, however (e.g., delta receptor selective compounds such as the enkephalin DADLE) do not distribute well to the CNS, and thus, may be important for peripheral effects.

Opioids are natural and synthetic drugs with morphine-like actions and include the opiates. Opioids are narcotic agonistic analgesics that produce drug dependence of the morphine type and are subject to control under federal narcotics law because of their addicting properties. Opiates are chemical agents derived from opium, such as, for example, morphine, codeine, and thebaine, with morphine being the most widely used derivative. The chemical classes of opioids with morphine-like activity include the purified alkaloids of opium consisting of phenanthrenes and benzylisoquinolines, semi-synthetic derivatives of morphine, phenylpiperidine derivatives, morphinan derivatives, benzomorphan derivatives, diphenyl-heptane derivatives, and propionanilide derivatives.

Physical dependence or drug addiction to narcotic drugs, for example, opioids, has been traditionally treated by drug withdrawal through administering an opioid antagonistic drug such as naltrexone or naloxone, withholding the opioid from the drug-dependent individual, gradually decreasing the amount of opioid taken by the individual over time, or substituting another opioid agonist, such as methadone, buprenorphine, or methadyl acetate, for the opioid to ameliorate the physical need for the opioid. When an opioid is discontinued, withdrawal symptoms appear, the character and severity of which are dependent upon such factors as the particular opioid being withdrawn, the daily dose of the opioid that is being withdrawn, the duration of use of the opioid, and the health of the drug dependent individual. The physical and psychological signs and symptoms associated with opioid withdrawal can be quite severe.

For example, the withdrawal of morphine, heroin, or other opioid agonists with similar durations of action from an individual dependent upon the opioid gives rise to lacrimation, rhinorrhea, yawning, and sweating 8 to 12 hours after the last dose of the opioid. As withdrawal progresses, the individual will be subject to dilated pupils, anorexia, gooseflesh, restlessness, irritability, and tremor. At the peak intensity of withdrawal, which is 48 to 72 hours for morphine and heroin, the individual suffers from increasing irritability, insomnia, marked anorexia, violent yawning, severe sneezing, lacrimation, coryza, weakness, depression, increased blood pressure and heart rate, nausea, vomiting, intestinal spasm, and diarrhea. The individual commonly experiences chills alternating with hot flushes and sweating, as well as abdominal cramps, muscle spasms and kicking movements, and pains in the bones and muscles of the back and extremities, and exhibits leukocytosis and an exaggerated respiratory response to carbon dioxide. Typically the individual does not eat or drink which, when combined with the vomiting, sweating, and diarrhea, results in weight loss, dehydration, and ketosis. The withdrawal symptoms from morphine and heroin usually disappear in 7 to 10 days, but the drug dependent individual suffers greatly during the withdrawal period.

Alternatively, if an opioid antagonistic drug is administered to the individual, such as naloxone or naltrexone, withdrawal symptoms develop within a few minutes after parenteral administration and reach peak intensity within 30 minutes, with a more severe withdrawal than from withholding the opioid. For example, naloxone is the current treatment of choice in cases of overdose. It is immediately effective but is encumbered by the precipitation of an intense withdrawal syndrome. Naltrexone can be used, for example, in maintenance therapy, but is quite aversive, which impedes wide acceptance and efficacy. Since addiction to cocaine and alcohol have been reported to also be mediated by specific opioid-sensitive brain cell networks (See, Gardner et al., Substance Abuse 2^{nd} Ed., pp. 70-99 (1992)) the use of opioid antagonists can be suitable for use in the treatment of alcohol and cocaine dependency. Thus, the opioid receptors can play a role in the dependency of multiple drug substances.

The use of opioid analgesics for the treatment of pain and during and/or after anesthesia can also lead to unwanted side effects, for example, respiratory depression. It is frequently necessary to titrate back or adjust the degree of analgesic/anesthesia in an individual receiving opioid pain management, for example, undergoing or recovering from a surgical procedure, due to complications associated with too high of a dose. The use of naltrexone and naloxone can produce undesirable withdrawal-like side effects such as pulmonary complications and gastrointestinal problems. Further, use of opioid analgesics for chronic pain can often be associated with constipation that can be a significant and limiting problem. There is currently no known treatment strategy available on the market to reduce the constipating effects of the opioid analgesics without blocking the analgesic effect and/or causing additional side effects (e.g., diarrhea and hyperalgesia).

Opiate antagonists further have utility when given alone in treating certain disorders that are not directly caused by opiate drugs. These include addiction to other drugs (e.g., nicotine, alcohol), compulsive behaviors, pruritis, irritable bowel syndrome, and more. As with the other applications, naltrexone and naloxone tend to display adverse effects that limit their utility.

As is known in the art the bone of a vertebrate is a dynamic tissue, with bone tissues essentially continuously being broken down and rebuilt. The metabolic processes of bone development occur during primary growth of the skeleton, continues through out the life of an individual and can be modulated by external factors, such as after an injury to the bone, and by hormonal changes that occur as well as during normal growth and development. Thus, bone metabolic development maintains the structural integrity of the skeleton and maintains a balance between the deposition and mobilization of minerals. Bone resorption and maintenance is mediated by osteoclast and osteoblast activity. Osteoclasts mediate bone resorption, whereas osteoblasts mediate bone building. Osteoclasts and osteoblasts regulate one another, and bone rebuilding takes place continuously for bone maintenance and repair. However, in circumstances where the balance of osteoblast and osteoclast activity is disrupted, abnormal bone loss or bone growth can take place. A number of metabolic and or developmental disorders can disrupt the metabolism of bone. Osteopenia and osteoporosis are diseases involving bone metabolic disorders characterized by a reduction in bone density. A World Health Organization committee defined four diagnostic categories: Normal, Osteopenia, Osteoporosis, and Established Osteoporosis. These categories depend on bone density and presence of fractures. In a particularly severe condition, the deposition of new bone is ineffective and the bone tissues break down faster than the body can repair leading to death of bone tissue, or osteonecrosis. When unsuccessfully treated, or left untreated, the osteonecrosis progresses, the bone structure may for instance, collapse, the joint surface may break down, or other negative effects occur, leading to pain, arthritis, or restriction in mobility.

Therefore, a need exists for agents which can be used in combination with opioids used in pain management to, for example reduce unwanted side effects at peripheral locations, in particular effects on bone, but which have reduced aversive properties and reduced propensity to precipitate withdrawal symptoms compared to existing compounds (e.g., naloxone, naltrexone and nalmefene) that readily cross the blood-brain barrier. Moreover, there exists a need for effective treatment of osteoporosis and osteopenia in patients not addicted to, or currently using opioid medication, with opioid receptors being a novel target.

For example, the treatment described herein can result in a reduction in the bone loss and negative effects on bone metabolism noted in individuals using opioids for long-term pain management (such as cancer patients) and in patients suffering from opioid drug dependency, while avoiding withdrawal symptoms and aversion encountered in when utilizing existing agents such as naloxone and naltrexone at doses that act as inverse agonists.

Accordingly, the present invention relates to a method for the treatment of bone loss, osteoporosis, osteopenia and other bone metabolic disorders in patients in need thereof comprising administering to the individual a therapeutically effect amount of a naloxone or naltrexone analog or a pharmaceutically acceptable salt thereof which is a peripheral antagonist at one or more of the opioid receptors.

### BRIEF SUMMARY OF THE INVENTION

The invention is embodied in the use of peripheral antagonists of the opioid receptors, such as the use of naltrexone and naloxone analogs, which can be opioid antagonist with peripheral selectivity at the μ opioid receptor, for the treatment of bone loss, osteoporosis, osteopenia and other bone disorders in individuals using opioid drugs, including patients using opioids for analgesia and in opioid drug-dependent individuals.

A further embodiment of the invention is the use of peripheral antagonists of the opioid receptors to modulate bone metabolism, in patients suffering from bone fractures, bone loss, osteoporosis, osteopenia, osteonecrosis, opioid-induced alteration of bone metabolism, and other bone metabolic disorders.

Consistent with predictions from basic principles of receptor pharmacology, the invention is further embodied in the administration of a therapeutically effective amount of the opioid, naloxone or naltrexone analogs described herein for the treatment of drug dependence, provides a beneficial result in reduction of undesirable effects on bone metabolism resulting from use of opioids. For example, the treatment described herein can result in a reduction in the bone loss and negative effects on bone metabolism noted in individuals using opioids for long-term pain management (such as cancer patients) and in patients suffering from opioid drug dependency, while avoiding withdrawal symptoms and aversion encountered in when utilizing existing agents such as naloxone and naltrexone.

Accordingly, the present invention relates to a method for the treatment of bone loss, osteoporosis, osteopenia and other bone metabolic disorders in patients in need thereof comprising administering to the individual a therapeutically effect amount of a naloxone or naltrexone analog or a pharmaceutically acceptable salt thereof which is a peripheral antagonist at one or more of the opioid receptors.

The naltrexone analogs suitable for use in the invention can be represented by Formula I and include the pharmaceutically acceptable salts thereof: 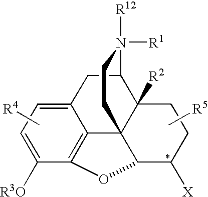
wherein:
R¹ and R¹² are H, alkyl, cycloalkyl(alkyl), for example, C₃-C₆ (cycloalkyl)alkyl, for example, C₃-C₆(cycloalkyl)methyl such as (cyclopropy)l methyl or C₅-C₇(cycloalkenyl)alkyl;
R² is H, OH or esters thereof, such as —OAc (O₂C(alkyl)), for example O₂(C₁-C₆ alkyl);
R³ is H, alkyl for example, C₁-C₆ alkyl, or (alkyl)C═O for example, ((C₁-C₆)alkyl)-C═O (acyl derivatives);
R⁴ and R⁵ are independently H, halogen (F, Cl, Br or I), alkyl, for example C₁-C₆ alkyl, alkoxy, such as C₁-C₄ alkoxy, nitro, amino, cyano, carboxyl or acyl which may be substituted for one or more hydrogens on the ring;
X is —R⁶—OR⁶, —NR⁷R⁸R⁹, —NCOR¹⁰, —NO₂, —R¹¹, —SR¹¹, wherein,
   R⁶ and R¹¹ are independently selected from alkyl, substituted alkyl, cycloalkyl, substituted cycloalkyl, aryl, substituted aryl, acyl, for example C₁-C₆ acyl such as —C(O)—C₁-C₆ alkyl or aroyl,
   R⁷, R⁸ and R¹⁰ are independently selected from hydrogen, alkyl, substituted alkyl, cycloalkyl, substituted cycloalkyl, aryl, or substituted aryl,
   R⁹ and R¹² can be present or absent and are independently selected from hydrogen, alkyl, substituted alkyl, cycloalkyl, substituted cycloalkyl, aryl, or substituted aryl
      and pharmaceutically acceptable salts thereof.

In one embodiment, the patient is being administered opioid drugs for the treatment of pain as part of an anesthetic regimen. In another embodiment, the individual is treated for chronic pain with an opioid drug, where the peripheral antagonist either blocks undesirable peripheral effects such as bone loss, osteopenia, constipation, or prevents abuse of the opiate, or a combination of such effects. In yet another embodiment, a drug-dependent individual is in therapy to reduce drug use or drug dependency and suffers from side effects of drug abuse. Moreover, peripheral opioid antagonists can serve in treatment of any medical conditions where opioid receptor overactivity at peripheral locations plays a pathological role, including disease such as bone loss, osteoporosis, osteopenia and other bone metabolic disorders in patients, and where the use of an antagonist (e.g., naloxone, naltrexone, nalmefene) is either contraindicated or is limited by side-effects associated with CNS activity.

The invention further relates to a method for the treatment of bone loss, osteoporosis, osteopenia and other bone metabolic disorders in patients in need thereof comprising administering to the individual a therapeutically effective amount of a sustained release composition comprising a biocompatible polymer and an effective amount of a naloxone or naltrexone analog or the pharmaceutically acceptable salts thereof which is peripheral antagonist at one or more of the opioid receptors, and in particular a peripheral antagonist at the μ opioid receptor. Use of a sustained release composition, as described herein, can be particularly desirable when a patient is being administered an opioid for the treatment of pain as part of an anesthetic regimen during long-term therapy.

The invention also relates to a kit, useful for the treatment of one or more of bone loss, osteoporosis, osteopenia and other bone metabolic disorders in patients in need thereof comprising a therapeutically effective dose of a naloxone or naltrexone analog, which is a peripheral antagonist at one or more of the opioid receptors, and in particular a peripheral antagonist at the μ opioid receptor, along with one or more of an opioid analgesic, drug delivery materials, and instructional materials associated with the doses. The kit is useful in the treatment of a patient who is being administered opioid drugs for the treatment of pain as part of an anesthetic regimen during long-term therapy.

### BRIEF DESCRIPTION OF THE DRAWINGS

For a fuller understanding, reference should be had to the following detailed description taken in connection with the accompanying drawings, in which:

FIG. 1 shows a graphical representation of bone rating over time following sarcoma injection with morphine compared to saline;

FIG. 1B shows a graphical representation of the percent of sarcoma induced mice with fractures at two time points, with morphine compared to saline;

FIG. 2A shows a graphical representation of osteoclast TRAP staining in sarcoma treated animals compared to control following morphine and saline infusion; and

FIG. 2B shows a graphical representation of IL-1B levels after sustained morphine infusion in sarcoma-induced compared to control.

### DETAILED DESCRIPTION OF THE INVENTION

The present invention relates to a method for the treatment of disease such as bone loss, osteoporosis, osteopenia and other bone metabolic disorders in patients in need thereof, or for the treatment of the side effects in patients using opioids such as bone loss, osteoporosis, osteopenia and other bone metabolic disorders that are responsive to opiate antagonists, comprising administering to the individual a therapeutically effect amount of a drug comprising a naloxone or naltrexone analog or a pharmaceutically acceptable salt thereof in a dose wherein said drug acts as a peripheral antagonist at one or more of the opioid receptors. One particular target for such a drug is the μ opioid receptor (mu opioid receptor, alternatively abbreviated as MOR).

The μ opioid receptor is probably the best-characterized opioid receptor in human, and exists in alternative forms or subclasses. The particular sublocalization of the individual opioid receptor classes and subclasses is not fully understood. Nonetheless, the μ opioid receptor is known to be present in the brain of mammals, along with being present in other tissues. The μ opioid receptor has a constitutively active state that may be represented as μ*. The μ opioid receptor is the main mediator of narcotic analgesia and addiction and can be classified as a G protein coupled receptor (GPCR) that is linked to an inhibition of adenylyl cyclase activity. While originally identified for their activation by opioids, the opioid receptors clearly have a number of crucial functions in the maintenance of homeostasis and reaction of the mammalian body to insult. The feature of basal level signaling activity in the opioid receptors is emerging as a recognized feature of a number of GPCRs, for example, the dopamine receptors, D1, D2 and D3, the adenosine receptor, the β2-adrenergic receptor, the serotonin receptor (5HT-2A) along with the opioid receptors. When opioid receptors are in the naive state (i.e. no prior exposure to exogenous or endogenous receptor activators), the activity of the μ* state is minimal, and most receptors are sensitive to opioid agonists (i.e. morphine). Recent findings indicate that the μ opioid receptor differs in its characteristics significantly between drug-naive and drug-tolerant/dependent states, with the constitutive or spontaneous activity of the μ opioid receptor being enhanced in the tolerant/dependent state. Moreover, spontaneous signaling by the μ. opioid receptor becomes sensitive to naloxone, naltrexone, and similar analogues, which act as inverse agonists by suppressing spontaneous activity of receptors in the dependent state.

The ∂ opioid receptor (DOR) is also a GPCR that is linked to an inhibition of adenylyl cyclase activity, thereby decreasing cAMP production and increasing intracellular potassium concentration and membrane hyperpolarization. Kappa opioid receptors are also GPCRs but the cellular effect is coupled to modulation of calcium concentration.

G protein-coupled receptors as a group display a basal signaling level in the absence of agonists. See Lefkowitz et al., (1993). In general, compounds which exhibit antagonistic behavior at a particular GPCR having basal signaling activity, for example the μ opioid receptor, can be categorized as either opioid antagonist with peripheral selectivity or inverse agonists based on the effect which they exhibit upon the basal signaling activity of the particular receptor for which they are a ligand following interaction. “Opioid antagonist with peripheral selectivity” are agents that block the effects of an agonist at the target receptor but do not significantly affect the level of spontaneous receptor activity. Opioid antagonist with peripheral selectivity do not affect basal signaling activity but still block agonist activity. See Milligan and Bond (1997). “Inverse agonists” are agents that block the effects of an agonist at the target receptor and also suppress spontaneous receptor activity. Thus, those compounds that suppress the basal level of signaling activity of the opioid receptor are referred to as inverse agonists. Thus, if a particular opioid receptor is involved in signaling cascades that regulate bone metabolism, and a basal level of receptor excitation is necessary for maintenance of bone metabolic homeostasis, for instance for balancing bone mineral deposition and bone mineral mobilization, a compound that suppresses basal activity is predicted to alter the metabolism of bone, potentially leading to bone disease. In the instance wherein basal level signaling is suppressing bone mineral deposition by osteoblasts, for instance, as may occur in age-related osteoporosis, disruption of the basal level signaling may provide relief from that bone metabolic disease.

Individual opioid drugs fall on a sliding scale of activity from full agonist to full inverse agonists. It appears possible that these pharmacological properties of a drug can change, however, during long-term stimulation. For example, the opioids naltrexone, naloxone, and nalmefene have been used for treating opioid overdose and opioid abuse/addiction, impulse control disorders, nicotine addiction certain forms of pruritis and a number of other psychiatric and gastrointestinal disorders. These compounds act as inverse agonists when utilized at the previously indicated concentrations, and when used as such can cause adverse peripheral effects in addition to blocking agonist activity. The prototypical opioid antagonists naloxone and naltrexone, while displaying neutral antagonistic behavior at an untreated μ opioid receptor, behave as inverse agonists at opioid-pretreated receptors, for example, at morphine pre-treated receptors. This switch in pharmacological effects at untreated or drug-pretreated receptors can be at least in part responsible for the severe withdrawal symptoms experienced by drug-dependent individual upon administration of naloxone and naltrexone.

An important contribution to the invention is embodied in the recognition that endogenous opioid peptides and opioid receptors are involved in controlling bone metabolism and that opioids and opioid receptors regulate bone physiology. The invention is further embodied in identification of peripheral antagonists as a method for countering the effects of long-term opioid use in disruption of bone metabolism. The invention is further embodied in the recognition of a correlation between patients receiving high doses of opioid agonists (chronic pain patients, methadone maintenance patients, and opioid addicts) and a greater risk of developing bone pathologies including osteopenia, osteoporosis and bone fractures. Methadone maintenance patients, for example, have a much greater incidence of osteopenia and osteoporosis than age matched controls. For instance, long-term users of morphine are at risk for the development of bone fractures and structural disruption of the bone. Furthermore, in a bone cancer model of pain, morphine dramatically increases bone density loss and fractures compared to untreated controls that have the same bone cancer There is increasing evidence that endogenous opioid peptides and opioid receptors play an important role in bone physiology and pathology. For instance, see Kim T W, et al., “Bone health in methadone maintenance treatment.” Paper presented at: CPDD (College on Problems of Drug Dependence) 65th Annual Meeting; June 2004; San Juan, Puerto Rico. Opioid receptors, for example, are found on cells responsible for bone formation and bone regulation.

Thus, it is a preferred embodiment of the invention that peripheral antagonists, useful for treating peripheral effects of opioid use, are predicted to provide therapeutic relief for disorders of bone metabolism, including, but not limited to bone metabolic disorders such as bone loss, osteopenia, osteoporosis, osteonecrosis, stress induced bone pain, and bone developmental disorders.

Peripherally restricted opioid antagonists (such as, for example the opioid antagonists with peripheral selectivity identified herein) would be an effective adjunct treatment for counteracting the adverse effects of opioid agonist therapy on bone metabolism and formation. Additional applications of opioid opioid antagonist with peripheral selectivity may be to prevent and or reverse dysfunctions of tooth formation and physiology. For additional information on the use of peripheral antagonists in modulating bone physiology, see:
Elvenes J, Andjelkov N, Figenschau Y, Seternes T, Bjorkoy G, Johansen O. Expression of functional mu-opioid receptors in human osteoarthritic cartilage and chondrocytes. Biochem Biophys Res Commun. 2003 Nov. 7; 311(1):202-7.
wilczek H, Stepan J. [Bone metabolism in individuals dependent on heroin and after methadone administration] Cas Lek Cesk. 2003; 142(10):606-8.
Langrod J, Lowinson J, Ruiz P. Methadone treatment and physical complaints: a clinical analysis. Int J Addict. 1981 July; 16(5):947-52.
Nilforoushan D, Shirazi M, Dehpour A R. The role of opioid systems on orthodontic tooth movement in cholestatic rats. Angle Orthod. 2002 October; 72(5):476-80.
Rosen H, Krichevsky A, Bar-Shavit Z. The enkephalinergic osteoblast. J Bone Miner Res. 1998 October; 13(10):1515-20.
Rosen H, Krichevsky A, Polakiewicz R D, Benzakine S, Bar-Shavit Z. Developmental regulation of proenkephalin gene expression in osteoblasts. Mol Endocrinol. 1995 November; 9(11):1621-31.
Rosen H, Bar-Shavit Z. Dual role of osteoblastic proenkephalin derived peptides in skeletal tissues. J Cell Biochem. 1994 July; 55(3):334-9.

Prior to the present application, it was not recognized that modulation of bone metabolism by opioid receptors is a crucial component of bone metabolism. Prior to the present application, the art generally teaches that deleterious or undesired effects on bone structure and metabolism as a result of opioid use is a “side-effect” of opioid use. The invention is embodied in the recognition that endogenous opioids may play a role in activating bone mobilization in response to pain. Thus, in a model according to the invention, expression of endogenous opioids in response to pain (i.e. pain resulting from bone damage or stress) both relieves pain, and increases bone mobilization. As the pain due to injury decreases, the opioid pain receptor activity decreases, and concomitantly bone mobilization decreases. As mobilization of bone tissues decreases, bone deposition is predicted to predominate, strengthening a remodeled bone structure. When exogenous opioids such as morphine are used in the treatment of pain, the balance of bone mobilization may be surprisingly shifted away from deposition resulting in weakening of the bone structure due to opioid receptor induced mobilization.

It is a common therapeutic strategy to provide opioids such as morphine to patients suffering from severe bone injuries. According to the embodiments of the invention, overuse of opioids may unnecessarily delay the full healing of bone injuries. In one study it has been shown that the opioid agonists Tramadol and Diclofenac have negative effects on the proliferation of osteoblasts, cells necessary for deposition of bone and healing of fractures. In vitro treatment of human osteoblasts with these drugs led to a concentration-dependent decrease of cell proliferation. Tramadol showed a significant decrease at a concentration of greater than 20 micrograms/ml. Although this concentration of opioid is approximately 10 times higher than the therapeutic concentration of 0.25 microgram/ml in serum, some effect on osteoblast activity at therapeutic concentrations is expected. Furthermore, Tramadol exhibits only weak opioid activity, so it is predicted that higher efficacy opioids (e.g., morphine, methadone and oxycodone) that are needed for more severe pain, will have incrementally greater effects on bone metabolism. The NSAID Diclofenac significantly decreased cell proliferation at a concentration of only 6 micrograms/ml, where the therapeutic concentration is approximately 1.5 micrograms/ml in serum. Thus, for Diclofenac significant decreases in the proliferation of human osteoblasts probably occurs at drug concentrations reached in vivo. This result may apply to all NSAIDs and suggest that another option for treating pain, at least in part, where the patient is at risk for bone loss or fractures (e.g., women and the elderly) is contraindicated by secondary effects on bone metabolism. This study demonstrates that the use opioids for treatment of bone injury and bone-associated pain may prolong healing of fractures. For further information, see: Matziolis G, Rau H M, Kiever P, Erli H J, Paar O. “[Modification of human osteoblasts by various analgesics]” Unfallchirurg. 2002 June; 105(6):527-31. Original in German. Thus, use of peripherally selective antagonists of the opioid receptor according to the invention, in conjunction with opioid agonists for pain management allows management of pain without negative effects on bone healing.

Metastasis of cancer to bone is the leading cause of pain in patients with malignant tumors. Such pain may be intense and unremitting, thus resulting in severe limitation of activity and a drastic decrease in the patient's quality of life. In the previous methods for the management of pain opioids have been the most common treatment for pain management in cancer patients reporting moderate to severe pain. The chronic nature of cancer pain often requires prolonged treatment with opiates. Surprisingly, prolonged use of opiates for pain management may induce a negative effect on bone metabolism by interfering with native signaling of the opioid receptors in bone tissue. Thus, opiate use by cancer patients while managing pain, may result in osteopenia and or osteoporosis such that bones are susceptible to fracture, pain inducing microfractures of the bone, or debilitation due increased bone pain. A further embodiment of the invention is an improvement wherein palliative opioid pain medication is provided for the advantage of pain management along with a peripheral opioid antagonist that serves to limit deleterious effects on bone in cancer patients and in other patients undergoing opioid therapy for pain.

In a murine bone cancer model in which sarcoma cells are injected and sealed into the femur, resulting in anatomical localization of the tumor, sustained morphine administration enhances sarcoma-induced pain associated with increased pronociception, necroplastic adaptations, accelerated and increased sarcoma induced bone loss, and accelerated time to fracture. Morphine administration did not alter tumor growth in vitro or tumor burden in vivo, suggesting that the opiate-induced increase in bone loss resulted from alterations in bone metabolism, consistent with this possibility, sarcoma-induced osteoclast activity and IL-1β proinflammatory cytokine that produces osteoclastogenesis and peripheral nociceptive sensitization were upregulated within the bone. The data suggest that management of bone cancer pain with opioids may induce unexpected risks including worsening of cancer pain and increasing bone destruction and indicate the need for conjuntive therapy combining opioids with compounds that counter opiate-induced effects on bone metabolism. The peripheral opioid antagonists according to the invention provide a conjunctive therapy for managing bone metabolism while opiates are in use. Specific data describing the metabolic effects of opiates on bone metabolism are provided in the examples.

Drug compounds embodied in the invention are those compounds that interfere with the interaction between opioids (i.e. opioid agonists) and the binding of these agonists to the peripheral opioid receptors. Preferred embodiments are drawn to those compounds that interfere with the disruption of osteoblast proliferation by opioid receptor agonists, and thus lead to increases in deposition of bone. Such compounds include peripherally selective opioid opioid antagonist with peripheral selectivity and protean antagonists or inverse agonists.

In practicing the use of peripheral antagonists for modulating bone metabolism, including for the treatment of diseases of the bone including, but not limited to bone loss, osteopenia, osteoporosis, stress fractures, and osteonecrosis, those compounds acting preferentially as peripherally selective antagonists are preferred, as action restricted to tissues remote from the central nervous system would allow the analgesic actions of the drug to remain intact while preventing the negative consequences of opioid agonists on bone metabolism. In certain embodiments, compounds that act as neutral antagonist are preferred, especially in situations where management of pain is a therapeutic consideration.

In other embodiments of the invention, for instance, where opioid receptors are naïve (i.e. no exogenous opioid agonists have recently been supplied) an inverse agonist may be appropriate, as a therapeutic requirement for maintaining the activity of exogenous opioids for pain relief is absent. Use of inverse agonists has been previously contraindicated or limited for those patients suffering from drug-dependency, whether such dependency is to opioids, alcohol, or nicotine, for instance. Known inverse agonists, particularly when supplied at doses where their primary activity is as an inverse agonist are predicted to have a greater potency compared to a neutral antagonist in limiting basal signaling of opioid receptors when those receptors are in an opioid-exposed state. As is known in the art, doses sufficient to induce inverse agonist activity in drug dependent patients also are more potent in the induction of adverse side effects.

Naltrexone is currently approved for the treatment of opioid addiction/abuse. Compliance with the pharmacotherapy has been poor, in part due to the side-effects associated with naltrexone therapy. Based on the work in our laboratories, perturbations of opioid basal signaling resulting from previous opioid exposure can be long-lived, especially after chronic, high dose exposure. The negative side-effects associated with naltrexone therapy are likely due in part to the inverse agonist nature of naltrexone. Opioid opioid antagonist with peripheral selectivity have the ability to reduce opioid craving and relapse, while being better tolerated than naltrexone in patients that have had significant previous exposure to opioids. For a review of the use of naltrexone in the treatment of addiction, see:
Tucker T, Ritter A, Maher C, Jackson H. Naltrexone maintenance for heroin dependence: uptake, attrition and retention. Drug Alcohol Rev. 2004 September; 23(3):299-309 (2004).

Given the known interactions between alcoholism and the endogenous opioid systems, it is likely that alcoholism and alcohol abuse result in increased basal activity at opioid receptors. With the recognition as part of the invention of the effects of opioid receptor agonists on the metabolism of bone, the invention is further embodied in a method for treating bone metabolic disorders using peripherally acting compounds according to the invention. Compounds delivered in doses where they act antagonistically including opioid opioid antagonist with peripheral selectivity, should provide adequate reduction of endogenous opioid activity sufficient to decrease negative effects on bone metabolism while not precipitating a withdrawal syndrome. The use of naloxone and naltrexone in the treatment of drug-dependent patients, which were prior to the invention prescribed at doses wherein their activity was primarily as an inverse agonist, is indicated at much lower doses to alleviate the negative effects on bone metabolism in patients.

By selectively blocking the peripheral opioid receptors, a preferred embodiment of the invention are those peripheral antagonists capable of limiting deleterious side-effects on bone metabolism associated with opioid therapy while preserving the analgesic (CNS mediated) effects of the agonists. Based on the structure activity and/or clinical profiles of the antagonists known to artisans and tested so far, certain opioid antagonist with peripheral selectivity actually act as inverse agonists when delivered to patients in the opioid-exposed state (protean ligands). This inverse agonist profile provides an explanation for the side-effects frequently reported in patients receiving existing drug compounds methylnaltrexone and Alvimopan and for the need to carefully titrate dose. Peripherally restricted opioid neutral antagonists of the invention (e.g., methylnaltroxone, naloxonmethiodide, and 69-naltrexamide) and thus only marginally affect the analgesic properties of opioid drugs. Yet these compounds would be delivered by the circulatory systems to peripheral locations, including the bone, wherein they these neutral peripheral antagonists could also be effective in limiting deleterious effects on bone metabolism associated with hyperactivity of endogenous opioid systems.

Where, in the past, clinicians would titrate the dose of inverse agonists such as naltrexone until withdrawal side effects occurred in order to signal that a sufficient dose to produce inverse agonism of opioid receptors had occurred, the invention is further embodied in teaching that compounds capable of acting peripherally can be delivered at a dose low enough to result in beneficial peripheral effects on bone, yet not at so high a dose as to substantially diminish the positive effects of pain relief provided by opioid agonists such as morphine. The compound, methylnaltrexone, for example, is predicted to be effective for decreasing deleterious effects on bone metabolism in patients with naïve receptors. Similarly compounds like 7-benzylidenenaltrexone (BNTX), acts as an inverse agonist in vitro at the mu and delta receptors and are expected to stimulate bone formation and potentially be useful in an opioid-naïve patient with osteopenia or osteoporosis.

Opioid antagonists such as naloxone and naltrexone are herein recognized as protean ligands. As such protean ligands are predicted to act primarily as inverse agonists when the system has been exposed to opioid drugs. Similarly, activity is predicted for opioid receptors exposed to high levels of endogenous opioids. These types of protean ligands act as opioid antagonist with peripheral selectivity of opioid receptors extant in the naïve state. Non-protean ligands such as compounds similar in structure to BNTX appear to act as inverse agonists under all circumstances, irrespective of whether the opioid receptors are naïve. Similar activity is predicted for the opioid inverse agonist Structure/Activity Relationships.

Previous pharmaceutical use of naloxone and naltrexone has been at doses where their activity was primarily as inverse agonists. Naloxone, naltrexone, nalmefene and peripherally acting analogs thereof, when supplied in a manner (for instance at low doses) that limits inverse agonistic activity, and limits their efficacy for the treatment of drug dependency provide a method of reducing the inhibitory activity of GPCR (opioid receptors). Thus, the invention is embodied in supplying neutral peripheral antagonists of the invention to individuals being provided with opiate analgesic drug therapy, thereby limiting negative side effects on bone metabolism. A preferred embodiment of the invention is a method of supplying the peripheral antagonists of the invention to cancer patients undergoing opioid therapy for pain management who are at risk for bone disorders precipitated by opioid use. In a preferred embodiment of the invention the narcotic antagonist is excluded from the brain, or alternatively, has reduced mobility to the CNS and brain relative to opioids employed for analgesic effects, a property of some of the compounds disclosed herein. In this manner, the peripheral antagonist activity does not interfere with pain treatment regimens.

Because of the differences in basal activity of the opioid receptors in the naïve state. conventional inverse agonists such as naltrexone, and naloxone, at doses too low to produce the pronounced inverse agonistic effect (such as the high dose for instantaneous induction of withdrawal) are indicated for use along with other peripheral opioid antagonists.

Extant art has disclosed the use of nalmefene at a relatively high concentration in the treatment of nicotine addiction. See Wang, et al., “Inverse agonists and opioid antagonist with peripheral selectivity at mu opioid receptor (MOR): possible role of basal receptor signaling in narcotic dependence.” J Neurochem, 77:1590-1600 (2001). The inverse agonistic effect of nalmefene at these doses is significant. Because the μ opioid receptor is a G protein-coupled receptor, a significant inverse agonist effect can translate into significant changes in cAMP concentrations. These changes in cAMP concentrations are known to play a role in development of dependence. See EJ Nestler and GK Aghajanian. “Molecular and cellular basis of addiction.” Science 278: 58-63 (1997). Nalmefene as used in alcohol dependence treatment regimes acts as an inverse agonist, not as a peripherally restricted antagonist, and the activity of nalmefene for the treatment of alcohol dependence demonstrates radically different efficacy. Other art discloses administration of derivatives of naloxone or naltrexone for the treatment of opiate drug abuse. Importantly, the previously utilized derivative structures were oximes of naloxone or naltrexone, with this structure implicating activity as inverse agonists. There is no indication that these compounds could be delivered with the pharmaceutical properties of the peripheral antagonists, because of conformational effects of the 6-double bond on the ring structure. Furthermore, there is no indication that these compounds just identified can act as peripherally restricted opioid antagonist with peripheral selectivity at the doses previously indicated. Double bonds at the C-6 position apparently change the stereochemistry of the ring structure to accentuate inverse effects. The pharmaceutical activity of prior art compounds acting as inverse agonists is thus distinct from that of the disclosed peripheral antagonists.

A series of oxymorphinan derivatives of naloxone and naltrexone are described with desirable neutral antagonist properties as disclosed in Sadee et al., U.S. Pat. No. 6,713,488. Within the series of oxymorphinans substitution at the C-6 position is critical to modification of activity. The reduced compounds of this series are derived from straight-forward structure-activity analysis of the relationship between modifications in the structure and those positions known to affect drug activity. In all cases analyzed, if the C-6 position is oxidized (i.e., SP2 hybridized, including as C═O and C═C), the resulting compound acts as an inverse agonist, at least in subjects in the morphine-dependent state. The compounds of Sadee et al., U.S. Pat. No. 6,713,488, are distinguished by a reduced C-6 position, containing substitutents such as OH, alkyl, amine/amide, and with no substitutent at the reduced C-6 position. The C-6 substitutents introduce additional properties including increased or reduced polarity at the C-6 position, or alternatively alter the ability of the substituted derivative to serve as a substrate for MDR1 (and hence act as a peripheral antagonist which is excluded from the CNS). All oxymorphinan derivatives of naltrexone and naloxone with a reduced C-6 position (i.e., SP3 hybridized) were found to act primarily as opioid antagonist with peripheral selectivity. Therefore, it is predicted that the C-6 reduced derivatives of naltrexone and naloxone will act as peripherally restricted antagonists. The synthetic scheme for the reduced naltrexone and naloxone derivatives is known to those skilled in the art of the organic chemistry of naltrexone and naloxone, and these compounds can be synthesized without undue experimentation, along with a variety of substitutions at positions other than the C⁶ position. Opioid antagonist with peripheral selectivity against receptors exhibiting spontaneous activity can be determined with the use of in vitro assays as known to those skilled in the art of pharmacology and chemistry. For example, the peptide CTAP was identified as a neutral antagonist by such assays, and is known to elicit significantly less withdrawal than naloxone in drug-dependent mice. See J. Pharm. Exper. Ther., 277: 484-190 (1996).

The invention is further embodied series of compounds provided by the determination that the C-6 substitutent conveys differential pharmacological behavior, in particular with respect to pharmacologic half-life of the compound and to the distribution and dissemination of the compound in the body of the patient, and importantly, on the class or subclass of the opioid receptor modulated by the peripheral antagonist. Thus, compared to C⁶H₂ compounds, the C⁶—OH compounds (i.e., 6-beta-naltrexol) are more polar and as a result have reduced penetration into the central nervous system (CNS) than are predicted for the C⁶H₂ compounds. Differences in solubility and penetration of the various compounds is advantageous for the generation of combination drug therapy compositions where the included neutral antagonist blocks peripheral effects only at low doses but would interfere with CNS effects at higher doses. Alternatively, the C-6-acetamide reduced naltrexone derivative, disclosed in U.S. Pat. No. 6,713,488, has a longer half-life in mice and is excluded, presumably by MDR1 transporter, from the brain. With respect to the selection of peripheral antagonists with preferential activity in bone tissues, an embodiment of the invention is the recognition that peripheral antagonists presumably have differential binding activity with respect to different classes and subclasses of opioid receptors. Wherein Sadee et al., U.S. Pat. No. 6,713,488, focused on the neutral antagonist effects upon the μ opioid receptor, in bone tissue, the effect on other classes of opioid receptor, including kappa and delta receptors of opioid use in patients is an important consideration.

Accordingly, the present invention relates to a method for the treatment of opioid-induced bone loss, osteoporosis, osteopenia and other bone disorders in individuals in need thereof comprising administering to the individual a therapeutically effective amount of an opioid antagonist or pharmaceutically acceptable salt thereof which is a peripheral antagonist at one or more of the opioid receptors. Furthermore, in an individual who is drug naïve (e.g., a patient who is not taking exogenous opioid analgesics) an opioid inverse agonist may also be used in the treatment of osteopenia and osteoporosis.

In a particular embodiment, the naltrexone analog is: 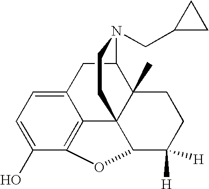
and the pharmaceutically acceptable salts thereof.

In a particular embodiment, the naloxone analog is 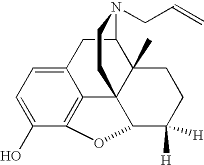
and pharmaceutically acceptable salts thereof.

As will be clear to those skilled in the relevant arts, the compounds of the invention are related to those compounds disclosed by Sadee et al., U.S. Pat. No. 6,713,488. The present invention is an improvement to a method for the treatment of bone loss, osteoporosis, osteopenia and other bone disorders.

The related naltrexone analogs disclosed by Sadee, et al., are represented by Formula III and include the pharmaceutically acceptable salts thereof: 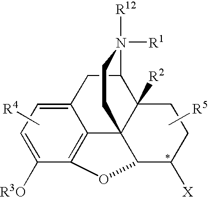
wherein:
R¹ is cycloalkyl(alkyl), for example, C₃-C₆ (cycloalkyl)alkyl, for example, C₃-C₆(cycloalkyl)methyl such as (cyclopropy)lmethyl or C₅-C₇(cycloalkenyl)alkyl;
R² is H, OH or esters thereof, such as —OAc (O₂C(alkyl)), for example O₂(C₁-C₆ alkyl);
R³ is H, alkyl for example, C₁-C₆ alkyl, or (alkyl)C═O for example, ((C₁-C₆)alkyl)-C═O;
R⁴ and R⁵ are independently H, halogen (F, Cl, Br or I), alkyl, for example C₁-C₆ alkyl, alkoxy, such as C₁-C₄ alkoxy, nitro, amino, cyano, carboxyl or acyl which may be substituted for one or more hydrogens on the ring;
X is —R⁶—OR⁶, —NR⁷R⁸R⁹, —NCOR¹⁰, —NO₂, —R¹¹, —SR¹¹
wherein,
   R⁶ and R¹¹ are independently selected from alkyl, substituted alkyl, cycloalkyl, substituted cycloalkyl, aryl, substituted aryl, acyl, for example C₁-C₆ acyl such as —C(O)—C₁-C₆ alkyl or aroyl,
   R⁷, R^{B} and R¹⁰ are independently selected from hydrogen, alkyl, substituted alkyl, cycloalkyl, substituted cycloalkyl, aryl, or substituted aryl,
   R⁹ and R¹² can be present or absent and are independently selected from hydrogen, alkyl, substituted alkyl, cycloalkyl, substituted cycloalkyl, aryl, or substituted aryl
      and pharmaceutically acceptable salts thereof.

Particular naltrexone analogs disclosed by Sadee, et al., are: 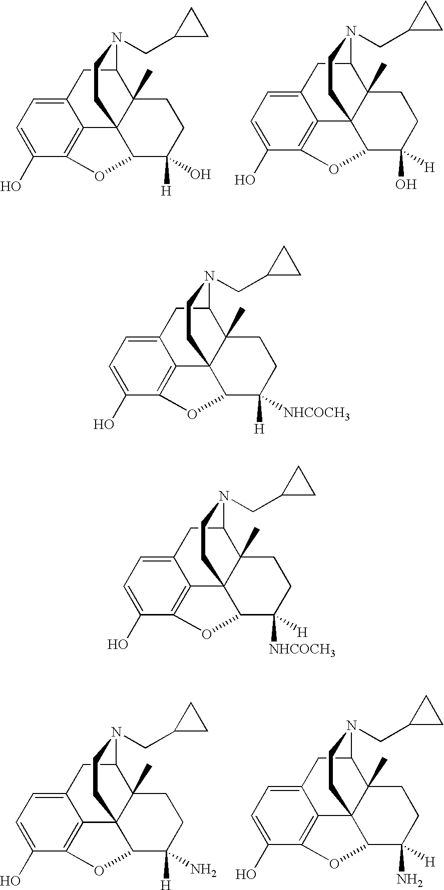
and the pharmaceutically acceptable salts thereof.

Particular naloxone analogs disclosed by Sadee, et al., are: 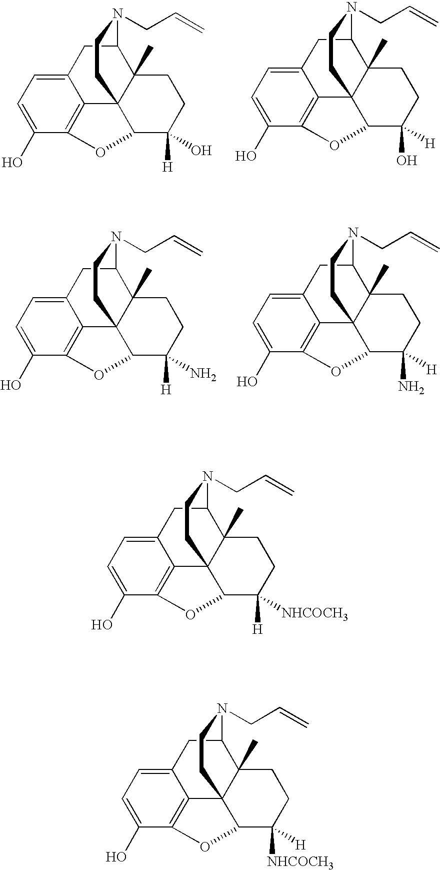
and pharmaceutically acceptable salts thereof.

Pharmaceutically acceptable salts of the naltrexone and naloxone analogs, which are peripheral antagonists at one or more of the opioid receptors, include salts derived from an appropriate base, such as an alkali metal (for example, sodium, potassium), an alkaline earth metal (for example, calcium, magnesium), ammonium and NX₄⁺ (wherein X is C₁-C₄ alkyl). Pharmaceutically acceptable salts of an amino group include salts of: organic carboxylic acids such as acetic, lactic, tartaric, malic, lactobionic, fumaric, and succinic acids; organic sulfonic acids such as methanesulfonic, ethanesulfonic, isethionic, benzenesulfonic and p-toluenesulfonic acids; and inorganic acids such as hydrochloric, hydrobromic, sulfuric, phosphoric and sulfamic acids. Pharmaceutically acceptable salts of a compound having a hydroxy group consist of the anion of said compound in combination with a suitable cation such as Na⁺, NH₄⁺, or NX₄⁺ (wherein X is for example a C₁₋₄ alkyl group).

In enantiomeric forms, compounds of the invention include individual enantiomers of the compounds of formula (I) in single species form substantially free of the corresponding enantiomer, as well as in admixture (in mixtures of enantiomeric pairs and/or in mixtures of multiple enantiomer species). In the compounds described herein the terms d and/anomers are used to distinguish the orientation of the substitutents at the chiral carbon marked with an (*).

As stated previously, opioids are natural and synthetic drugs with morphine-like actions and include the opiates. The chemical classes of opioids with morphine-like activity are the purified alkaloids of opium consisting of phenanthrenes and benzylisoquinolines, semi-synthetic derivatives of morphine, phenylpiperidine derivatives, morphinan derivatives, benzomorphan derivatives, diphenyl-heptane derivatives, and propionanilide derivatives. The principal phenanthrenes are morphine, codeine, and thebaine. The principal benzoisoquinolines are papaverine, a smooth muscle relaxant, and noscapine. Semi-synthetic derivatives of morphine include diacetylmorphine (heroin), hydromorphone, oxymorphone, hydrocodone, apomorphine, etorpine, and oxycodone. Phenylpiperidine derivatives include meperidine and its congeners diphenoxylate and loperamide, alphaprodine, anileridine hydrochloride or phosphate, and piminodine esylate. Morphinan derivatives include levorphanol. The diphenyl-heptane derivatives include methadone and its congeners, and propoxyphene. Propionanilide derivatives include fentanyl citrate and its congeners sufenil citrate and alfenatil hydrochloride.

The opioid antagonists naloxone, naltrexone and nalmefene are approved by the Food and Drug administration for a number of indications including the reversal of opioid agonist actions, the treatment of heroin addiction and for alcoholism. These compounds have also been tested clinically and pre-clinically for many other indications including pruritis, metabolic disorders and various psychiatric disorders. Several newer opioid antagonists that have selectivity for blocking peripheral versus central opioid receptors have also been brought forward through phase II and phase III clinical trials.

While the efficacy of opioid antagonist therapy in some of the aforementioned conditions/diseases is firmly established, what is not widely appreciated is that the approved opioid antagonists when delivered at the indicated concentrations all act as inverse agonists at the mu opioid receptor when the receptors have been exposed to opioid agonists, or endogenous conditions have led to enhanced endorphin activity which also stimulates μ opioid receptors and increases basal activity at the μ opioid receptor. It has been demonstrated in preclinical models that endogenous or exogenous opioids can increase constitutive or basal signaling at opioid receptors. Additional clinical data suggests that increased constitutive or basal signaling at opioid receptors similarly occurs in humans treated acutely or chronically with opioid analgesics, or with condition where the endogenous opioid system is physiologically activated.

For veterinary applications, opioids are often used in the management of pain following injury, for instance following injury to racing horses and other performing animals. When injuries involve injuries to bone, or when opioids are delivered for long-term pain management, the invention is further embodied in supplying peripheral opioid antagonists as an adjunct to palliative opioid therapies. Thus, pain can be managed, along with limiting damage to bone or avoiding deleterious modification of bone metabolism by providing a therapeutically effective amount of a peripheral antagonist along with an opioid agonist. The peripheral action of compounds according to the invention thus provides for a new method of protecting bone from injury, and for moderating the side effects on bone metabolism from opioid use. In a specific example, certain high value animals, such as thoroughbred racehorses, or performing animals may sustain an injury to the bone that would be sufficiently severe that the animal might be destroyed if pain cannot be managed effectively. Unfortunately, prior to the invention, opioid pain management would have the effect of slowing recovery of bone strength. The use of peripheral antagonists according to the invention thus allows management of pain while maximizing opportunity for bone recovery.

The invention also relates to a kit, useful for the treatment of one or more of bone loss, osteoporosis, osteopenia and other bone metabolic disorders in patients in need thereof comprising a therapeutically effective dose of a naloxone or naltrexone analog, which is a peripheral antagonist at one or more of the opioid receptors, and in particular a peripheral antagonist at the μ opioid receptor, along with one or more of an opioid analgesic, drug delivery materials, and instructional materials associated with the doses. The kit is useful in the treatment of a patient is being administered opiate drugs for the treatment of pain as part of an anesthetic regimen during long-term therapy, patients undergoing treatment for osteoporosis, individuals suffering from osteonecrosis, and individuals receiving opioids for pain management.

For terms not otherwise defined herein:

“a” or “an” refers to one or more;

“patient” or “individual” as that term is used herein refers to the recipient of the treatment described herein and includes individuals in long-term therapy to prevent relapse to drug use, individuals who have taken an overdose of a drug and are in need of acute treatment, individuals who are undergoing active withdrawal treatment from addiction and infants born to drug addicted mothers. Mammalian and non-mammalian patients are included. In a specific embodiment, the patient is a mammal, such as a human, equine, canine, murine, feline, bovine, ovine, swine or ursine. In a preferred embodiment, the patient is a human. It is understood that the drug-dependent individual can be dependent upon multiple drugs;

a “therapeutically effective amount” refers to the amount of the naltrexone or naloxone analog or sustained release composition having the naltrexone or naloxone analog incorporated therein, needed to elicit the desired biological response following administration. The desired biological response herein can be sufficient blockade of the μ opioid receptor resulting in alleviation of drug dependency, modulation of pain management or reducing adverse effects associated with current pain management such as diarrhea and constipation. Therapeutically effective amounts of the peripheral antagonists can be formulated as pharmaceutically suitable compositions (e.g. in the form of pharmaceutically acceptable salts). A therapeutically effective amount can be in the range of about 1 microgram (μg) to about 100 milligrams (mg) per kilogram of body weight of the recipient per day. For example, from about 5 μg to about 75 mg per kilogram body weight per day, such as from about 10 μg to about 50 mg per kilogram body weight per day. The administered dose can be present as two or more sub-doses administered at appropriate intervals throughout the day. Alternatively, if the condition of the patient requires, the doses can be administered as continuous infusion.

The compositions of this invention can be administered in vivo, for example, to an individual in need of therapy, for example, a human, or an animal. In a preferred embodiment, the naltrexone and naloxone analogs, which are peripheral antagonists at one or more of the opioid receptors can also be administered peripherally and in large part be retained in the peripheral circulation (e.g., in the bloodstream). Peripheral antagonists are designed to have potent peripheral activity but reduced central activity. Administration can be accomplished orally, or parenterally such as by injection, implantation (e.g., subcutaneously, intramuscularly, intraperitoneally, intracranially, and intradermally), administration to mucosal membranes (e.g., intranasally, intravaginally, intrapulmonary, buccally or by means of a suppository), or in situ delivery (e.g., by enema or aerosol spray) to provide the desired dosage of opioid, or naltrexone or naloxone analog to treat bone disorders or modulate undesirable effects of narcotic analgesic (such as osteopenia or osteoporosis) in the treatment of pain or anesthesia, in an individual in need thereof.

“Peripheral antagonists” as that term is used herein refers to agents that block the effects of an agonist at target opioid receptor in tissues not part of the central nervous system. Under certain conditions peripheral antagonists, as distinct from opioid antagonist with peripheral selectivity, may have an effect the level of spontaneous activity present at the target receptor. Certain peripheral antagonists are restricted to peripheral activity and excluded from activity on the central nervous system.

“Opioid antagonist with peripheral selectivity” as that term is used herein, refers to agents that block the effects of an agonist at target opioid receptor but do not significantly affect the level of spontaneous activity present at the target receptor. “Neutral antagonist at the μ opioid receptor” as that term is used herein refers to an agent which can bind selectively to the resting, drug-sensitive μ opioid receptor state, to the constitutively active μ opioid receptor state, or to both, blocking the effects of an agonist at the receptor, but not significantly effecting the level of spontaneous activity present at the receptor. “Opioid antagonist with peripheral selectivity” as that term is used herein, refers to agents that block the effects of an agonist at the target receptor, but do not significantly affect the level of spontaneous activity present at the target receptor. “Neutral antagonist at the μ opioid receptor” as that term is used herein refers to an agent which can bind selectively to the resting, drug-sensitive p opioid receptor state, to the constitutively active μ opioid receptor state, or to both, blocking the effects of an agonist at the receptor, but not significantly effecting the level of spontaneous activity present at the receptor.

“Partial inverse agonists”, as that term is used herein refers to agents which block the affects of an agonists at the target receptor and also suppress spontaneous receptor activity at the target receptor.

“Full inverse agonist” as that term is used herein refers to an agent that suppresses completely spontaneous receptor activity at the target receptor and will also block the affects of an agonist at the target receptor.

“Partial agonists” as that term is used herein refers to agents that induce an agonist response, for example, receptor activation, but even at maximal dosages result in only partial activation of the resting, drug-sensitive target receptor.

The naloxone and naltrexone analogs represented by the structures presented herein can be synthesized using standard synthetic procedures such as those described in March J., Advanced Organic Chemistry, 3^{rd} Ed. (1985). Employing, for example, naltrexone or naloxone as the starting material. Many of the analogs of naltrexone and naloxone which possess neutral antagonist activity at the μ opioid receptor, for example, the analogs wherein the 6-keto functionality has been reduced to an —OH functionality are known compounds, and their syntheses have been described, for example, by Chatterjie et al., J. Med. Chem., 18, pp. 490-492 (1975) and Jiang et al., J. Med. Chem., 20, pp. 1100-1102 (1977

As used herein, in reference to the present invention, the term “alkyl” is intended to be broadly construed as encompassing: (i) alkyl groups of straight-chain as well as branched chain character; (ii) unsubstituted as well as substituted alkyl groups, wherein the substitutents of substituted alkyl groups may include any substitutents which are compatible with such alkyl and which retain the peripherally antagonistic behavior of the naloxone and naltrexone analogs. Examples of substitutents for substituted alkyl groups include halogen, for example, fluoro, chloro, bromo and iodo, amino, amido, C₁-C₄ alkyl, C₁-C₄ alkoxy, nitro, hydroxy; (iii) saturated alkyl groups as well as unsaturated alkyl groups, the later including groups such as alkenyl substituted alkyl, for example, allyl, methallyl, propallyl, butenylmethyl, etc. Alkynyl substituted alkyl groups and any other alkyl groups containing unsaturation which is compatible with such alkyl groups and which retains the antagonistic behavior of the naloxone and naltrexone analogs; and (iv) alkyl groups including linking or bridge moieties, for example, heteroatoms such as nitrogen, oxygen, sulfur.

As used herein, in reference to the present invention, the term aryl is intended to be broadly construed as referring to carbocyclic, for example, phenyl, naphthyl as well as heterocyclic aromatic groups, for example pyridyl, thienyl, furanyl and encompassing unsubstituted as well as substituted aryl groups, wherein the substitutents of the substituted aryl groups can include any substitutents which retain the peripheral antagonistic behavior of the naloxone and naltrexone analogs. Examples of substitutents for substituted aryl groups include one or more of halogen, for example, fluoro, chloro, bromo and iodo, amino, amido, C₁-C₄ alkyl, C₁-C₄ alkoxy, nitro, trifluoromethyl, hydroxy, hydroxyalkyl containing a C₁-C₄ alkyl moiety, etc.

For example, the kit can comprise a container containing a suitable neutral antagonist and, in addition, the kit can include instructional materials containing directions (for example, dosage protocols) for the administration of the pharmaceutically effective compositions described here along with contraindications. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to Internet sites that provide such instructional materials.

In another embodiment, the invention relates to a method for the treatment of bone disorders in an individual in need thereof comprising administering to the individual a therapeutically effect amount of a sustained release composition comprising a biocompatible polymer; and an effective amount of a naloxone or naltrexone analog or the pharmaceutically acceptable salts thereof which is a peripheral antagonist at one or more of the opioid receptors.

The term “sustained release composition” as defined herein, can comprise a biocompatible polymer having incorporated therein at least one naloxone or naltrexone analog which is a peripheral antagonist at one or more of the opioid receptors. Suitable biocompatible polymers, can be either biodegradable or non-biodegradable polymers or blends or copolymers thereof, as described herein.

Typically, the sustained release composition can contain from about 0.01% (w/w) to about 50% (w/w) of the naloxone or naltrexone analog which is a neutral antagonist at the μ opioid receptor (dry weight of composition). The amount of naloxone or naltrexone analog used will vary depending upon the condition of the patient, the desired effect of the agent, for example, to treat active withdrawal or to prevent relapse in long-term therapy, the planned release levels, and the time span over which the agent will be released. A preferred range of agent loading is between about 0.1% (w/w) to about 30% (w/w) agent. A more preferred range of agent loading is between about 0.5% (w/w) to about 20% (w/w) agent.

The sustained release compositions of this invention can be formed into many shapes such as a film, a pellet, a rod, a filament, a cylinder, a disc, a wafer or a microparticle. A microparticle is preferred. A “microparticle” as defined herein, comprises a polymer component having a diameter of less than about one millimeter and having a naltrexone or naloxone analog which is a neutral antagonist at the p opioid receptor dispersed therein. A microparticle can have a spherical, non-spherical or irregular shape. Typically, the microparticle will be of a size suitable for injection. A preferred size range for microparticles is from about one to about 180 microns in diameter.

As defined herein, a sustained release of a naltrexone or naloxone analog of the present invention is a release of the agent from a sustained release composition. The release occurs over a period which is longer than that period during which a therapeutically significant amount of the naloxone or naltrexone analog, would be available following direct administration of a solution of the analog. The period of sustained release can be, for example, about one day, about two days, about seven days, about ten days or more as needed to attain the desired results. It is preferred that a sustained release be a release of naloxone or naltrexone analog, which is a peripheral antagonist at one or more of the opioid receptors, which occurs over a period of greater than two days. A sustained release of a naltrexone or naloxone analog of the invention, from a sustained release composition can be a continuous or a discontinuous release, with relatively constant or varying rates of release. The continuity of release and level of release can be affected by the type of polymer composition used (e.g., monomer ratios, molecular weight, and varying combinations of polymers), agent loading, and/or selection of excipients to produce the desired effect.

The polymers of the sustained release composition described herein are biocompatible. Suitable biocompatible polymers, can be either biodegradable or non-biodegradable polymers or blends or copolymers thereof, as described herein.

Suitable biocompatible polymers, can be either biodegradable or non-biodegradable polymers or blends or copolymers thereof, as described herein. A polymer is biocompatible if the polymer and any degradation products of the polymer are non-toxic to the recipient and also possess no significant deleterious or untoward effects on the recipient's body, such as an immunological reaction at the injection site.

“Biodegradable”, as defined herein, means the composition will degrade or erode in vivo to form smaller chemical species. Degradation can result, for example, by enzymatic, chemical and physical processes. Suitable biocompatible, biodegradable polymers include, for example, poly(lactides), poly(glycolides), poly(lactide-co-glycolides), poly(lactic acid)s, poly(glycolic acid)s, polycarbonates, polyesteramides, polyanydrides, poly(amino acids), polyorthoesters, poly(dioxanone)s, poly(alkylene alkylate)s, copolymers or polyethylene glycol and polyorthoester, biodegradable polyUrethane, blends thereof, and copolymers thereof.

Suitable biocompatible, non-biodegradable polymers include non-biodegradable polymers selected from the group consisting of polyacrylates, polymers of ethylene-vinyl acetates and other acyl substituted cellulose acetates, non-degradable polyUrethanes, polystyrenes, polyvinylchloride, polyvinyl fluoride, poly(vinyl imidazole), chlorosulphonate polyolefins, polyethylene oxide, blends thereof, and copolymers thereof.

Acceptable molecular weights for polymers used in this invention can be determined by a person of ordinary skill in the art taking into consideration factors such as the desired polymer degradation rate, physical properties such as mechanical strength, and rate of dissolution of polymer in solvent. Typically, an acceptable range of molecular weight is of about 2,000 Daltons to about 2,000,000 Daltons.

In a particular embodiment, the polymer is biodegradable polymer or copolymer. In a more preferred embodiment, the polymer is a poly(lactide-co-glycolide)(hereinafter “PLG”). The PLG can have a lactide:glycolide ratio, for example, of about 10:90, 25:75, 50:50, 75:25 or 90:10 and a molecular weight of about 5,000 Daltons to about 70,000 Daltons.

It is understood that when the naltrexone or naloxone analog, which is a peripheral antagonist of one or more of the opioid receptors, is incorporated into a biocompatible polymer for sustained release of the analog, the sustained release composition can include additional components which can stabilize the analog and/or modify the release profile of the naltrexone or naloxone analog from the sustained release composition. That is, the naltrexone or naloxone analog of the sustained release composition can be stabilized against loss of potency and/or loss of activity, all of which can occur during formation of the sustained release composition having the naltrexone or naloxone analog dispersed therein, and/or prior to and during in vivo release of the analog. In addition, the period of release of the naltrexone or naloxone analog can be prolonged.

A suitable excipient or a specific combination of excipients can be employed in the sustained release composition. “Excipient”, as that term is used herein, is any agent which binds or interacts in a covalent or non-covalent manner or is included with the naloxone or naltrexone analog in the sustained release composition.

Suitable excipients include, for example, carbohydrates, amino acids, fatty acids, surfactants, and bulking agents, and are known to those skilled in the art. An acidic or a basic excipient is also suitable. The amount of excipient used is based on ratio to the naltrexone or naloxone analog, on a weight basis. For amino acids, fatty acids and carbohydrates, such as sucrose, trehalose, lactose, mannitol, dextran and heparin, the ratio of carbohydrate to analog, is typically between about 1:10 and about 20:1. For surfactants the ratio of surfactant to analog is typically between about 1:1000 and about 2:1. Bulking agents typically comprise inert materials. Suitable bulking agents are known to those skilled in the art.

The excipient can also be a metal cation component which acts to modulate the release of the naltrexone or naloxone analog. A metal cation component used in modulating release typically comprises at least one type of multivalent metal cation. Examples of metal cation components suitable to modulate release include or contain, for example, Mg(OH)₂, MgCO₃ (such as 4MgCO₃.Mg(OH)₂.5H₂O), MgSO₄, Zn(OAc)₂, Mg(OAc)₂, ZnCO₃ (such as 3Zn(OH)₂2ZnCO₃)ZnSO₄, ZnCl₂, MgCl₂, CaCO₃, Zn₃(C₆H₅O₇)₂ and Mg₃(C₆H₅O₇)₂. A suitable ratio of metal cation component to polymer is between about 1:99 to about 1:2 by weight. The optimum ratio depends upon the polymer and the metal cation component utilized. A polymer matrix containing a dispersed metal cation component to modulate the release of an agent from the polymer matrix is further described in U.S. Pat. No. 5,656,297 to Bernstein et al. the teachings of which are incorporated herein by reference in their entirety.

A number of methods are known by which sustained release compositions (polymer/active agent matrices) can be formed. In many of these processes, the material to be encapsulated is dispersed in a solvent containing a wall forming material. At a single stage of the process, solvent is removed from the microparticles and thereafter the microparticle product is obtained.

Methods for forming a composition for the sustained release of biologically active agent are described in U.S. Pat. No. 5,019,400, issued to Gombotz et al., and issued U.S. Pat. No. 5,922,253 issued to Herbert et al. the teachings of which are incorporated herein by reference in their entirety.

In this method, a mixture comprising a biologically active agent, a biocompatible polymer and a polymer solvent is processed to create droplets, wherein at least a significant portion of the droplets contains polymer, polymer solvent and the active. These droplets are then frozen by a suitable means. Examples of means for processing the mixture to form droplets include directing the dispersion through an ultrasonic nozzle, pressure nozzle, Rayleigh jet, or by other known means for creating droplets from a solution.

Means suitable for freezing droplets include directing the droplets into or near a liquified gas, such as liquid argon or liquid nitrogen to form frozen microdroplets which are then separated from the liquid gas. The frozen microdroplets are then exposed to a liquid or solid non-solvent, such as ethanol, hexane, ethanol mixed with hexane, heptane, ethanol mixed with heptane, pentane or oil.

The solvent in the frozen microdroplets is extracted as a solid and/or liquid into the non-solvent to form a polymer/active agent matrix comprising a biocompatible polymer and a biologically active agent. Mixing ethanol with other non-solvents, such as hexane, heptane or pentane, can increase the rate of solvent extraction, above that achieved by ethanol alone, from certain polymers, such as poly(lactide-co-glycolide) polymers.

A wide range of sizes of sustained release compositions can be made by varying the droplet size, for example, by changing the ultrasonic nozzle diameter. If the sustained release composition is in the form of microparticles, and very large microparticles are desired, the microparticles can be extruded, for example, through a syringe directly into the cold liquid. Increasing the viscosity of the polymer solution can also increase microparticle size. The size of the microparticles which can be produced by this process ranges, for example, from greater than about 1000 to about 1 micrometers in diameter.

Yet another method of forming a sustained release composition, from a suspension comprising a biocompatible polymer and a biologically active agent, includes film casting, such as in a mold, to form a film or a shape. For instance, after putting the suspension into a mold, the polymer solvent is then removed by means known in the art, or the temperature of the polymer suspension is reduced, until a film or shape, with a consistent dry weight, is obtained.

### EXAMPLES

The following Examples provide demonstration as to how the present invention may be practiced, but should not be construed as limiting.

### Example 1 Sustained Morphine Accelerates Sarcoma-Induced Bone Loss and Fracture

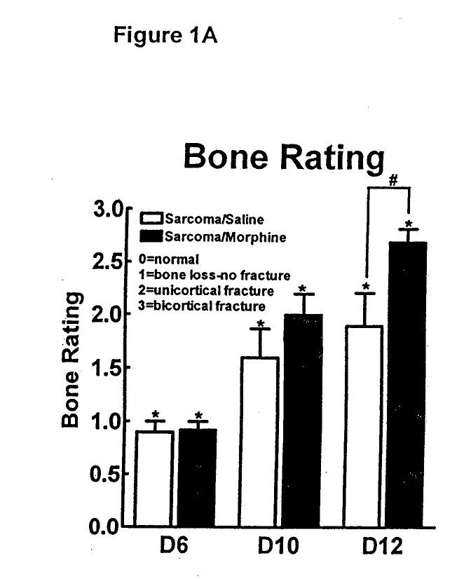

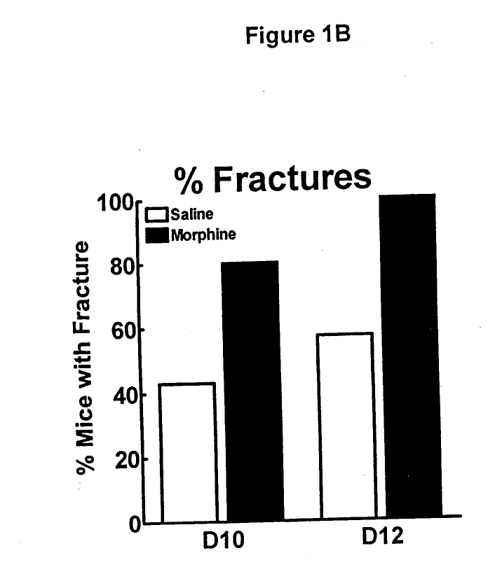

To determine the effects of sustained morphine exposure on sarcoma-induced bone loss, radiographic images were taken following behavioral testing. Radiographs of bones 12 days following femoral injection of sarcoma or control medium 15 days into morphine or saline infusion show that sustained morphine administration increased sarcoma-induced bone loss. In the sarcoma treated mice with saline infusion, bone loss was observed in the distal head of the bone and extended along the femur to the proximal head. Radiographs were rated according to a 3 point scale by an experimenter blinded to the experimental condition of the femur. Ratings of sarcoma treated mice with saline or morphine infusions 6, 10, and 12 days following sarcoma injection show that some sarcoma-induced bone loss is observed 6 days following, with no difference between morphine or saline treated mice. Unicortical fractures begin to develop 10 days following sarcoma injection in both morphine and saline treated mice. Mice receiving morphine infusion across 5 days demonstrated more bone destruction 12 days following sarcoma injection compared with saline treated mice, with more mice showing unicortical fractures. The most severe bone loss was observed in the distal third of the femur in all conditions. In sarcoma treated mice with morphine infusion, there was a dramatic increase in bone destruction in both the proximal and distal heads of the femur. Visual ratings of the radiographs by an observer blinded to the experimental conditions show that bone loss is observed by 6 days following femoral injection of sarcoma cells. As shown in FIG. 1A, the graph shows means +/−SEM.* indicates significant difference from control saline group, p<0.05 indicates significant difference between saline and morphine treated mice within the sarcoma or the control groups, p<0.05. The pre-morphine bone loss was equivalent between groups, indicating no baseline group differences of bone loss prior to morphine and saline infusion. The sarcoma-induced bone loss increased in both morphine and saline treated mice in a time-dependent manner (*p<0.05). Sustained morphine exposure enhanced sarcoma-induced bone loss compared to sarcoma-treated mice receiving saline infusion, with a significant increase in sarcoma-induced bone loss in morphine infused animals by 12 days following femoral injection, 5 days into infusion (#* p<0.05). Sustained morphine administration also doubled the incidence of sarcoma-induced fracture at 10 days and 12 days following sarcoma injection into the femur, indicated by full thickness cortical bone loss. Moreover, a count of the mice showing fractures 10 and 12 days following femoral injection of sarcoma shows that sustained morphine exposure approximately doubles the fracture rate at both time-points (FIG. 1A). These data indicate that sustained morphine exposure not only enhances sarcoma-induced pain, but also increases the sarcoma-induced bone loss resulting in a dramatic increase in fracture rate. An increase in bone loss and fracture rate could, and likely does, contribute to the morphine-induced pain in these animals. However, the sustained morphine-induced necroplastic changes within the primary afferent fibers and spinal cord in combination with the sarcoma-induced necroplastic changes within the pain pathways also likely play an important role in the sustained morphine-induced increased in sarcoma-induced pain mediators. Indeed, the pronociceptive necroplastic changes induced by the sustained morphine exposure likely increases fracture-evoked pain in the sarcoma-treated mice that develop fractures. Nonetheless as shown in FIG. 1B, the percent of sarcoma treated mice with fractures (unicortical or bicortical) 10 and 12 days following sarcoma injection. Morphine infusion doubled the percent of animals showing fractures compared to saline infused animals.

### Example 2

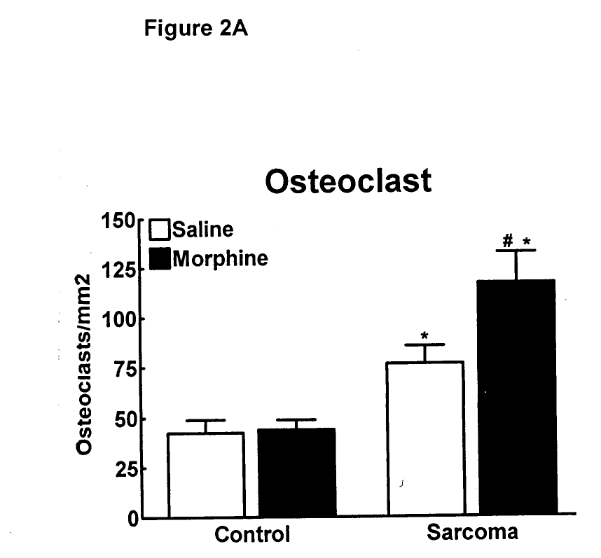

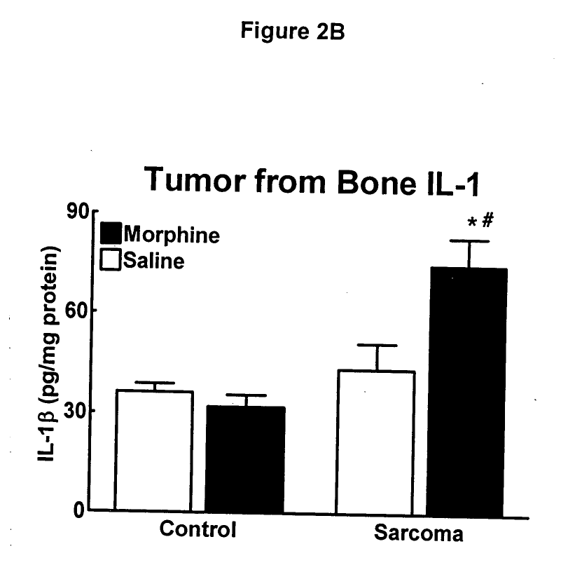

Previous studies have shown that osteolytic cancers, such as the sarcoma cell line used in these studies, upregulate osteoclasts within the bone. To determine whether the enhanced morphine-induced bone loss is mediated through further upregulation of osteoclasts, osteoclasts were stained and counted within the metaphysis of the distal head of the femur using tartrate resistant acid phosphatase (TRAP) staining. Osteoclast staining was significantly increased in sarcoma treated animals compared to control animals (*p>0.05); FIG. 2A). There was no difference in osteoclast staining in control animals treated with morphine as compared to those treated with saline, suggesting that sustained morphine itself did not alter osteoclastogenesis. However, sustained morphine infusion increased sarcoma-induced upregulation of osteoclasts compared to saline infusion, suggesting that sustained morphine increases sarcoma-induced upregulation of osteoclastogenesis (*p<0.05; FIG. 2B).

While the invention has been described with reference to preferred embodiments, those skilled in the art will understand that various changes may be made and equivalents may be substituted without departing from the scope of the invention. In addition, many modifications may be made to adapt a particular situation or material to the teachings of the invention without departing from its essential scope. It is intended that all matter contained in the above description or shown in the accompanying drawings be interpreted as illustrative and not in a limiting sense. Therefore, the invention is not to be limited to any particular embodiment disclosed as the best mode contemplated for carrying out this invention, rather the invention will include all embodiments falling within the scope of the appended claims. In this application all units are in the metric system and all amounts and percentages are by weight, unless otherwise expressly indicated. All citations referred herein are expressly incorporated herein by reference. Unless otherwise defined, all terms are considered to be defined according to Webster's New Twentieth Century Dictionary Unabridged Second edition.

We claim:

1. A method for the treatment of a bone metabolic disorder in an individual in need thereof, comprising administering to the individual a therapeutically effective amount of an opioid antagonist with peripheral selectivity.

2. The method of claim 1, wherein the opioid antagonist is represented by Formula I: 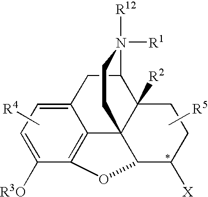
wherein:
R¹ and R¹² are H, alkyl, cycloalkyl(alkyl), for example, C₃-C₆ (cycloalkyl)alkyl, for example, C₃-C₆(cycloalkyl)methyl such as (cyclopropy)l methyl or C₅-C₇(cycloalkenyl)alkyl;
R² is H, OH or esters thereof, such as —OAc (O₂C(alkyl)), for example O₂(C₁-C₆ alkyl);
R³ is H, alkyl for example, C₁-C₆ alkyl, or (alkyl)C═O for example, ((C₁-C₆)alkyl)-C═O (acyl derivatives);
R⁴ and R⁵ are independently H, halogen (F, Cl, Br or I), alkyl, for example C₁-C₆ alkyl, alkoxy, such as C₁-C₄ alkoxy, nitro, amino, cyano, carboxyl or acyl which may be substituted for one or more hydrogens on the ring;
X is —R⁶—OR⁶, —NR⁷R⁸R⁹, —NCOR¹⁰, —NO₂, —R¹¹, —SR¹¹, wherein,
R⁶ and R¹¹ are independently selected from alkyl, substituted alkyl, cycloalkyl, substituted cycloalkyl, aryl,
substituted aryl, acyl, for example C₁-C₆ acyl such as —C(O)—C₁-C₆ alkyl or aroyl,
R⁷, R⁸ and R¹⁰ are independently selected from hydrogen, alkyl, substituted alkyl, cycloalkyl, substituted cycloalkyl, aryl, or substituted aryl,
R⁹ and R¹² can be present or absent and are independently selected from hydrogen, alkyl, substituted alkyl, cycloalkyl, substituted cycloalkyl, aryl, or substituted aryl
and pharmaceutically acceptable salts thereof.

3. The method of claim 1, wherein the individual is in long-term opioid therapy for pain management.

4. The method of claim 1, wherein the opioid receptor is one or more of a mu opioid receptor, a delta opioid receptor, and a kappa opioid receptor.

5. The method of claim 1, wherein the opioid receptor is a delta opioid receptor.

6. The method of claim 1, wherein the bone metabolic disorder is one or more of fractures, bone loss, osteoporosis, osteopenia, osteonecrosis, and opioid-induced alteration of bone metabolism.

7. The method of claim 6, wherein the bone metabolic disorder is osteoprosis.

8. A method for the treatment of bone metabolic disease in an individual in need thereof comprising administering to the individual a therapeutically effect amount of a sustained release composition comprising:
a. biocompatible polymer; and
b. an effective amount of an opioid antagonist with peripheral selectivity selected from a naloxone analog or naltrexone analog or the pharmaceutically acceptable salts thereof which are a peripheral antagonist at one or more of the opioid receptors.

9. The method of claim 8, wherein the sustained release composition releases a therapeutically effective amount of the neutral antagonist for about 7 days.

10. A method for the treatment of bone loss, osteoporosis, osteopenia and other bone metabolic disorders in patients in need thereof comprising administering to the individual a therapeutically effect amount of an agent comprising one or more of an opioid analog, a naloxone analog and a naltrexone analog or a pharmaceutically acceptable salt thereof wherein said agent is a peripheral antagonist at one or more of the opioid receptors.

11. The method of claim 10, wherein the patient is one or more of a patient using opioid drugs, a patient using opioids for analgesia and an opioid drug-dependent patient.

12. The method of claim 11, wherein the patient using opioids for analgesia is a patient receiving opioids for treatment of cancer related pain.

13. The method of claim 2, wherein the individual is a long term user of opioids.

14. The method of claim 1 wherein a peripheral antagonist is given together with an opiate analgesic.

15. The method of claim 1, wherein the individual is undergoing opioid analgesia treatment and wherein a opioid antagonist with peripheral selectivity is given to modulate peripheral side effects of analgesia.

16. The method of claim 1, wherein the compounds act peripherally when administered peripherally.

17. The method of claim 1, wherein the compounds act peripherally when administered orally.

18. A method for the treatment of opioid drug interactions in a patient resulting from increased activity of the endogenous opioid systems comprising administering to the individual a therapeutically effective amount of a naloxone analog or naltrexone analog or a pharmaceutically acceptable salt thereof which is a peripheral antagonist at one or more of the opioid receptors.

19. The method of claim 18 wherein the patient is suffering from one or more of bone loss, osteoporosis, osteopenia, osteonecrosis, and opioid-induced alteration of bone metabolism.

20. The method of claim 19 wherein the patient is suffering from opioid-induced alteration of bone metabolism.
